(19) United States
(12) Patent Application Publication
(10) Pub. No.: US 2010/0062084 A1
(43) Pub. Date: Mar. 11, 2010

(54) Treatment Of Gastrointestinal Diseases

(75) Inventors: Hayley Bennett, Stockport (GB); Ian Stanley Roberts, Cheshire (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: THE UNIVERSITY OF MANCHESTER, Manchester (GB)

(21) Appl. No.: 12/513,214

(22) PCT Filed: Nov. 2, 2007
(86) PCT No.: PCT/GB2007/004180

§ 371 Date: Oct. 7, 2009

(30) Foreign Application Priority Data
Nov. 13, 2006 (GB) 0622136.0

(51) Int. Cl. A61K 36/81; A61K 31/192; A61K 31/702; A61K 31/70; A61P 1/00; A61P 1/04; A61P 35/00

(52) U.S. Cl. 424/725; 514/570; 514/54; 514/23

(57) ABSTRACT

The present invention relates to compositions (including medicaments and nutritional products) for use in the prevention or treatment of gastrointestinal disease. Such compositions comprise a therapeutically effective amount of a less than or equal to 4 kDa soluble fraction derivable from potato juice.

The present invention relates to the prevention and treatment of gastrointestinal diseases and in particular, to the prevention and treatment of gastrointestinal diseases with extracts derived from edible plants.

Gastritis covers a variety of different conditions that all cause inflammation of the stomach. Inflammation may result from an infection with bacteria but other factors, including traumatic injury and regular use of certain pain relievers, can also contribute to gastritis.

In spite of the many conditions associated with gastritis, the signs and symptoms of the disease are very similar: a burning pain in the upper abdomen and occasionally, bloating, belching, nausea or vomiting. In some cases, gastritis can lead to ulcers and an increased risk of stomach cancer.

A peptic ulcer is a sore on the lining of the stomach or duodenum. One cause of peptic ulcer is bacterial infection, but some ulcers are caused by long-term use of nonsteroidal anti-inflammatory agents (NSAIDs), like aspirin and ibuprofen. In a few cases, cancerous tumors in the stomach or pancreas can cause ulcers.

Helicobacter pylori (H. pylori) is a type of bacteria that is a major cause of chronic gastritis and plays a pivotal role in the development of both gastric and duodenal ulcers (1). H. pylori weakens the protective mucous coating of the stomach and duodenum, which allows acid to get through to the sensitive lining beneath. Both the acid and the bacteria irritate the lining and cause a sore, or ulcer.

H. pylori is able to survive in stomach acid because it secretes enzymes that neutralize the acid. This mechanism allows H. pylori to make its way to the “safe” area; the protective mucous lining. Once there, the spiral shape of the bacterium helps it burrow through the lining. Chronic infection with this organism is associated with increased risk for the development of gastric adenocarcinoma and gastric lymphoma (2). Adhesion of H. pylori to the gastric epithelium has been shown to influence disease severity (3). It promotes translocation of a cytotoxin-associated antigen A (CagA) into the host cell, via the cag pathogenicity island encoded type IV secretion system (TFSS), causing subversion of host cell functions and increasing the risk of gastric cancer development (4). As such, the ability to adhere to host gastric mucosa is a prerequisite for both initial colonisation and continued survival and proliferation with the subsequent development of host pathology.

Treatment of H. pylori infections usually follows a seven or ten day triple therapy regime, including a proton pump inhibitor such as omeprazole or rabeprazole, together with commonly used antibiotics such as clarithromycin, metronidazole and amoxicillin (5). Although effective, such a regime can be undesirable from both a cost perspective and also as adverse side effects such as diarrhoea, metallic taste and allergic reactions are known to occur. Resistance to clarithromycin and metronidazole is also a major cause of treatment failure with 10 to 20% of patients failing to successfully eradicate the organism following therapy (6). This high failure rate has lead to a number of non-antibiotic therapies being considered as potential alternatives for treatment of H. pylori infection (7).

Several reports have described the activity of plant extracts and their isolated constituents against H. pylori. For example the use of vegetables such as broccoli for the treatment of gastritis is well known (8). In addition the active constituent of broccoli, the isothiocyanate sulforaphane, showed activity against H. pylori including antibiotic resistant strains (9). Furthermore the inhibition of growth and urease production of H. pylori by alcoholic extracts of the shallot Allium ascalonicum (10) and by oregano and cranberry (11) has also been shown. Other plants that have been shown to exhibit toxicity towards H. pylori are the North American herbal medicines Sanguinaria canadensis and Hydrastis Canadensis (12), the Asian herb Plumbago zeylanica (13), a range of Greek traditional medicines (14), and green tea extract (15). More recently a report described the antibacterial activity of nutmeg, parsley, tarragon, long pepper, sage and cinnamon against H. pylori (16). However, many of these culinary and medicinal herbs exhibit a broad spectrum of activity with toxicity having been shown against both Gram-positive and Gram-negative bacteria (17). Thus the consumption of such herbs and their products may also have adverse effects on the probiotic micro flora, many of which are known to be beneficial (18). Therefore, in order to be efficacious, natural anti-H. pylori agents should exhibit a narrow spectrum of activity and ideally be selective towards H. pylori alone.

It is an object of the present invention to provide alternative non-antibiotic compositions useful in the prevention or treatment of gastrointestinal diseases, in particular gastric and duodenal ulcers.

According to a first aspect of the present invention there is provided a composition for use in the prevention or treatment of gastrointestinal diseases comprising a therapeutically effective amount of a less than or equal to 4 kDa soluble fraction derivable from potato juice.

According to a second aspect of the present invention there is provided the use of a less than or equal to 4 kDa soluble fraction derivable from potato juice for the manufacture of a medicament for the prevention or treatment of gastrointestinal diseases.

According to a third aspect of the present invention there is provided a method for the treatment of gastrointestinal diseases comprising administering to a subject in need of such treatment a therapeutically effective amount of a less than or equal to 4 kDa soluble fraction derivable from potato juice.

More preferably, the fraction is less than or equal to 3 kDa, especially being 1 and 3 kDa. Most especially, the soluble fraction derivable from potato juice comprises a therapeutically effective amount of equal to or greater than 500 Da to less than or equal to 1 kDa.

In particular, the fraction may include an at least one oligosaccharide. Preferably the fraction is neutral at pH 8. The fraction preferably includes phenol glycosides, phenol aglycones and/or nitrogenous compounds, preferably being other than alkaloids. The fraction may include one or more amino acids selected from the group consisting of threonine, valine and leucine or isoleucine or methionine.

According to a fourth aspect of the present invention there is provided a soluble fraction derivable from potato juice comprising an amount of equal to or greater than 500 Da to less than or equal to 1 kDa wherein the soluble fraction comprises three caffeic acid derivatives and two aromatic derivatives.

It is to be appreciated that the soluble extract according to the present invention may be in pure form or alternatively it may also be mixed with other compounds, provided those compounds do not inhibit the anti-adhesive properties of the extract according to the invention.

The less than or equal to 4 kDa extract, more specifically, the equal to or greater than 500 Da to less than or equal to 1 kDa soluble fraction has been found to be especially effective in the inhibition of the binding of H. pylori and the removal of bound bacteria and thus have efficacy for preventing or treating gastritis and duodenal ulcers. Without wishing to be bound by any particular theory, it is believed that the less than or equal to 4 kDa fraction, more specically, the equal to or greater than 500 Da to less than or equal to 1 kDa soluble fraction, may either mimic or compete with bacterial receptors that enable the bacteria to adhere to gastric or duodenal epithelium, although the Inventor does not wish to be bound by this hypothesis.

The less than or equal to 4 kDa soluble extract, including the equal to or greater than 500 Da to less than or equal to 1 kDa soluble fraction, according to the invention may be prepared in its simplest form by homogenising potato juice and decanting off the aqueous supernatant, centrifuging and fractionating the supernatant to separate off the desired fraction. This extract may be concentrated, enriched or condensed by, for example, standard techniques, for example evaporation under reduced pressure. Examples of concentrates are those which are at least 2-fold concentrated, more usually, at least 4-fold, for example at least 8-fold, or at least 20-fold, or at least 100-fold, or at least 200-fold, or at least 1000-fold.

Any suitable method may be used to fractionate the aqueous solution into the active fraction, including, for example, molecular weight filtration, chromatography on a suitable solid support such as sepharose gel or fast protein liquid chromatography (FPLC) which may be based on size exclusion chromatography.

The less than or equal to 4 kDa fraction, including the equal to or greater than 500 Da to less than or equal to 1 kDa soluble fraction according to the present invention may be prepared from potato juice by one or more of the following steps:

(a) maceration of potato tuber, optionally freshly peeled; and

(b) centrifugal filtration of the resulting aqueous juices.

Appropriate organic solvents may be used to aid purification of the fraction, as may chromatographic procedures, such as ion exchanger resin and further size exclusion, for example on Superdex™.

In a preferred embodiment of the present invention the fraction is subjected to ion exchange chromatography to partially purify the extract. Preferably, the fraction is subjected to an anion HPLC fractionation at pH 8 with the flow-through being subjected to cation HPLC at pH 8. The flow-through from the cation HPLC is collected to produce a partially purified extract having the desired anti-adhesive properties.

The fractionated supernatant may optionally be freeze or spray dried to make a powdered fraction according to the invention. A crude preparation (re the fractionated supernatant mentioned above) may be used substantially “neat” or even diluted. When this is the case, the supernatant (whether diluted or not) may be mixed with a number of other agents that may be added for nutritional reasons, medical reasons or even for the purposes of adjusting the palatability of the extract for consumption by the subject being treated.

For instance, the extract may be formulated with a food or drink product to provide a functional food having the characteristic that it contains less than or equal to 4 kDa, more preferably less than or equal to 3 kDa, and most especially that is contains an equal to or greater than 500 Da to less than or equal to 1 kDa soluble fraction of potato juice that prevents or relieves the symptoms of gastritis. For example, the extract may be formulated with a diary product using milk as the raw material (for example milk, a milk shake, yoghurt, yoghurt drink or dairy supplement) to produce a palatable drink/beverage with the added benefit that it contains the active fraction and therefore will be highly suitable as a refreshment for sufferers of gastritis. The functional food product may comprise a probiotic drink. It is to be appreciated that the extract could be formulated as an alternative beverage, such as a fruit juice, albeit it is likely that such a choice would be less desirable to a sufferer of gastritis.

Alternatively, the crude preparation may be included in a nutritional liquid for enteral feeding. For instance, the active fraction may be mixed with saline or an aqueous solution (other vitamins, minerals and nutrients may be included) for enteral feeding of subjects.

It will be appreciated that concentration of the crude preparation may be required or alternatively a powder composition may be desired. When this is the case the crude extract will need to be concentrated/dehydrated.

The less than or equal to 4 kDa fraction, more preferably the less than or equal to 3 kDa fraction and the especially equal to or greater than 500 Da to less than or equal to 1 kDa soluble fraction may be formulated as a powder, granule or semisolid for incorporation into capsules. For presentation in the form of a semisolid, the extract can be dissolved or suspended in a viscous liquid or semisolid vehicle such as a polyethylene glycol, or a liquid carrier such as a glycol, for example propylene glycol, or glycerol or a vegetable or fish oil, for example an oil selected from olive oil, sunflower oil, safflower oil, evening primrose oil, soya oil, cold liver oil, herring oil, etc. This may then be filled into capsules of either the hard gelatine or soft gelatine type or made from hard or soft gelatine equivalents, soft gelatine or gelating-equivalent capsules being preferred for viscous liquid or semisolid fillings.

Powders comprising the less than or equal to 4 kDa fraction, the more preferably less than or equal to 3 kDa fraction and the especially equal to or greater than 500 Da to less than or equal to 1 kDa soluble fraction according to the invention are particularly useful for making pharmaceutical or nutritional products that may be used to prevent or treat gastrointestinal diseases, in particular gastric and duodenal ulcers and gastric and duodenal carcinomas.

Freeze-drying or spray drying represent preferred methods for producing a powder comprising the extract according to the invention. Spray drying results in free-flowing granular powder mixes with good flow properties and quick dissolving characteristics.

Powdered extract may be reconstituted as a clear/translucent low viscosity drink/beverage. Reconstitution may be into water, dairy or fruit juices as discussed above. It will be appreciated that the powder may be packaged in a sachet and reconstituted as a drink by a subject when required or desired.

Powder mixes represent preferred embodiments of the invention. Such mixes comprise powdered extract (as described above) mixed with further ingredients. Such ingredients may be added for nutritional or medical reasons or for improved palatability. The powdered extract may be mixed with granulated sugars of varying particle sizes to obtain free-flowing powder mixes of varying sweetness.

Alternatively natural sweeteners or artificial sweeteners (for example aspartame, saccharin and the like) may be mixed with the powdered extract for reconstitution as a low calorie/reduced calorie sweetened drink. The powder mix may comprise a mineral supplement. The mineral may be any one of calcium, magnesium, potassium, zinc, sodium, iron, and their various combinations.

Powder mixes may also contain buffering agents such as citrate and phosphate buffers, and effervescent agents formed from carbonates, for example bicarbonates such as sodium or ammonium bicarbonate, and a solid acid, for example citric acid or an acid citrate salt.

The less than or equal to 4 kDa, more preferably the less than or equal to 3 kDa, and especially the equal to or greater than 500 Da to less than or equal to 1 kDa soluble fraction can be presented as food supplements or food additives, or can be incorporated into foods, for example functional foods or nutriceuticals. Such products may be used as staple foods as well as under circumstances where there may be a clinical need.

The powders may be incorporated in to bakery or cereal products, such as snack food bars for example fruit bars, nut bars and cereal bars. For presentation in the form of snack food bars, the powder can be admixed with any one or more ingredients selected from dried fruits such as sundried tomatoes, raisins and sultanas, ground nuts or cereals such as oats and wheat.

It will be appreciated that the less than or equal to 4 kDa fraction, the less than or equal to 3 kDa fraction, and especially the equal to or greater than 500 Da to less than or equal to 1 kDa soluble fraction may advantageously be formulated as a pharamaceutical for use as a medicament (requiring a prescription or otherwise).

The powdered fraction or concentrated liquid fraction may also be incorporated into tablets, lozenges, sweets or other food-stuffs for oral ingestion. It will also be appreciated that such powdered fraction or concentrated liquid fraction may be incorporated into slow-release capsules or devices which may be ingested and are able to release the fraction into the intestines over a long period of time.

The less than or equal to 4 kDa fraction, the less than or equal to 3 kDa fraction, and especially the equal to or greater than 500 Da to less than or equal to 1 kDa soluble fraction may also be microencapsulated. For instance encapsulation may be by calcium-alginate gel capsule formation. Kappa-carrageenan, gellan gum, gelatin and starch may be used as excipients for micro-encapsulation.

Crude preparations, liquid concentrates, powders and the like may be combined with known therapeutic agents for treating gastritis. As such the extract according to the invention may be used in a very effective combination therapy. It will be appreciated that the extract in solution may act as an ideal vehicle for other therapeutic agents for treating gastritis.

The less than or equal to 4 kDa fraction, the less than or equal to 3 kDa fraction, and especially the equal to or greater than 500 Da to less than or equal to 1 kDa soluble fraction may also be included in combination/synbiotic therapies that include a probiotic portion. The bacteria contained within many probiotic mixtures do not possess adhesive properties so would not be affected by the inclusion of the active fraction.

The compositions of the present invention can be presented in the form of unit dosage forms containing a defined concentration of the extract. Such unit dosage forms can be selected so as to achieve a desired level of biological activity.

The amount of an extract required by a subject is determined by biological activity and bioavailability that in turn depends on the formulation, mode of administration, the physicochemical properties of the extract and whether it is being used as a monotherapy or in a combined therapy. For example, a daily dose for a human adult may be between 0.1 g and 100 g of freeze-dried or spray-dried powder (however formulated). More preferably being between 1 g and 30 g, for example about 5 g, 10 g, or 15 g as required.

The extract is particularly useful when included in pharmaceutical formulation such as a tablet or a capsule. Such formulations may be required to be enterally-coated if bioavailabilty dictates this. Known procedures, such as those conventionally employed by the pharmaceutical industry (for example in vivo experimentation, clinical trials etc), may be used to establish specific formulations of pharmaceutical compositions and precise therapeutic regimes (such as daily doses of the compounds and the frequency of administration).

It will be appreciated that conventional “nutriceutical” procedures may be employed to create liquid drinks, powder mixes and food-stuffs comprising the less than or equal to 4 kDa fraction, more preferably a less than or equal to 3 kDa fraction and especially the equal to or greater than 500 Da to less than or equal to 1 kDa soluble fraction.

Daily doses may be given as a single administration (for example a daily tablet for oral consumption or as a single liquid drink). Alternatively the extract used may require administration twice or more times during a day. As an example, a 100 ml orange drink containing between 0.1 to 20 g of spray dried extract, preferably between 0.3 to 10 g of spray dried extract and more preferably between 0.5 to 3.0 g may be used to quench thirst at regular intervals throughout the day and thereby deliver a recommended dose.

It will be appreciated that nutritional products supplemented with the less than or equal to 4 kDa potato extract, the less than or equal to 3 kDa fraction and especially the equal to or greater than 500 Da to less than or equal to 1 kDa soluble fraction represent an ideal means for providing subjects with, or at risk of developing, gastritis or other gastrointestinal disease with the extract according to the present invention.

Therefore, according to a fifth aspect of the present invention there is provided a nutritional product for use in the prevention or treatment of gastrointestinal diseases comprising a therapeutically effective amount of a less than or equal to 4 kDa fraction, or a less than or equal to 3 kDa fraction and especially an equal to or greater than 500 Da to less than or equal to 1 kDa soluble fraction derivable from potato juice.

The nutritional product may comprise:
(a) a clear, low viscosity, water-like, stable, ready-to-use, bottled, carbonated or non-carbonated drink; or a concentrated clear liquid for reconstitution containing a less than or equal to 4 kDa fraction or, a less than or equal to 3 kDa fraction or especially an equal to or greater than 500 Da to less than or equal to 1 kDa soluble fraction derivable from potato juice;
(b) a powder/granular mix to be reconstituted with water or any other orally ingestible liquid as a drinkable liquid, containing a less than or equal to 4 kDa fraction or a less than or equal to 3 kDa fraction or especially an equal to or greater than 500 Da to less than or equal to 1 kDa soluble fraction derivable derivable from potato juice; or
(c) a powder or granular mix mixed into a food stuff (for example a cereal or chocolate bar, lozenge or the like).

The nutritional product may be as described above and may or may not contain water soluble vitamins, additional mineral supplements, nutritional compounds, antioxidants or flavourings.

The present invention will be further illustrated, by way of examples, with reference to the accompanying drawings in which:

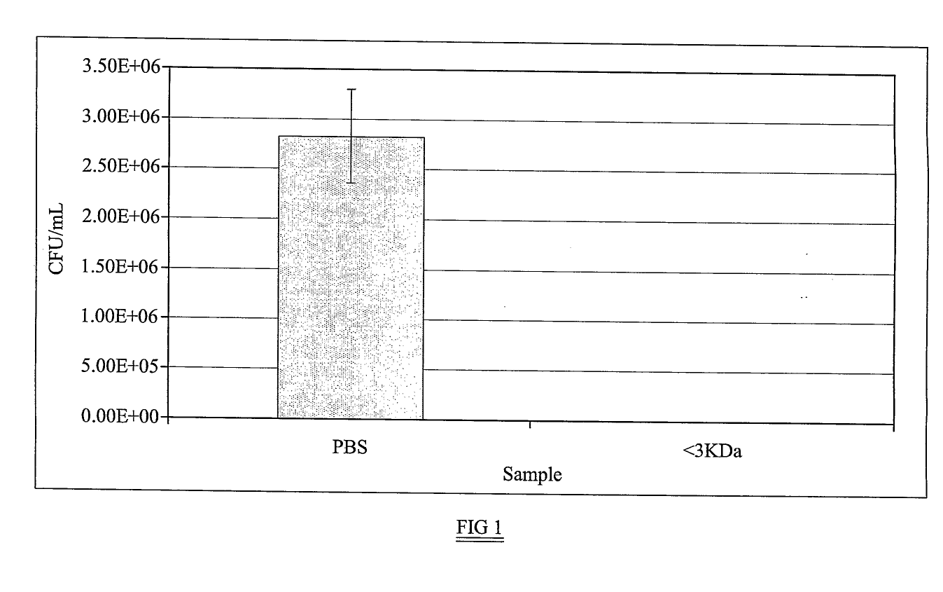

FIG. 1 is a graph showing that the anti-adhesive properties of potato juice (PJ) are located in a less than 3 kDa fraction;

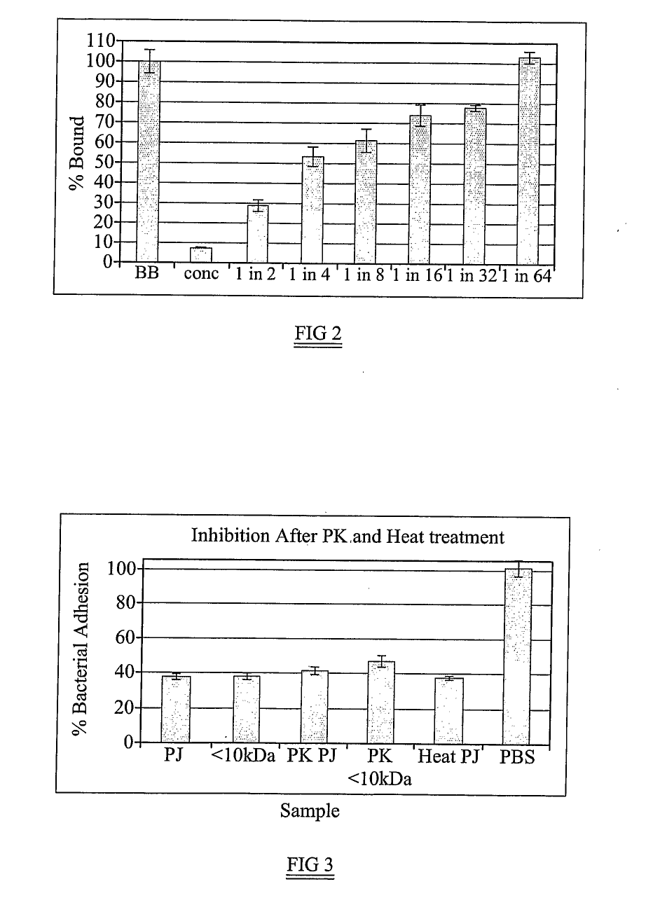

FIG. 2 is a graph showing that the anti-adhesive activity of the less than 3 kDa fraction is a dose dependent effect;

FIG. 3 illustrates the effect of proteinase K and heat treatment on the anti-adhesive properties of potato juice (PJ) and fractions thereof;

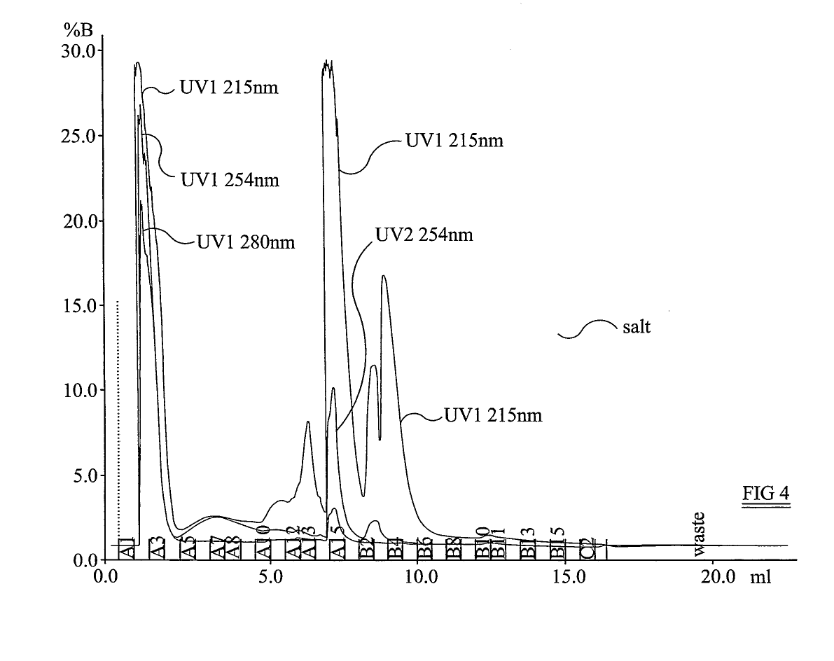

FIG. 4 is an anion exchange HPLC separation chromatogram of the less than or equal to 3 kDa fraction;

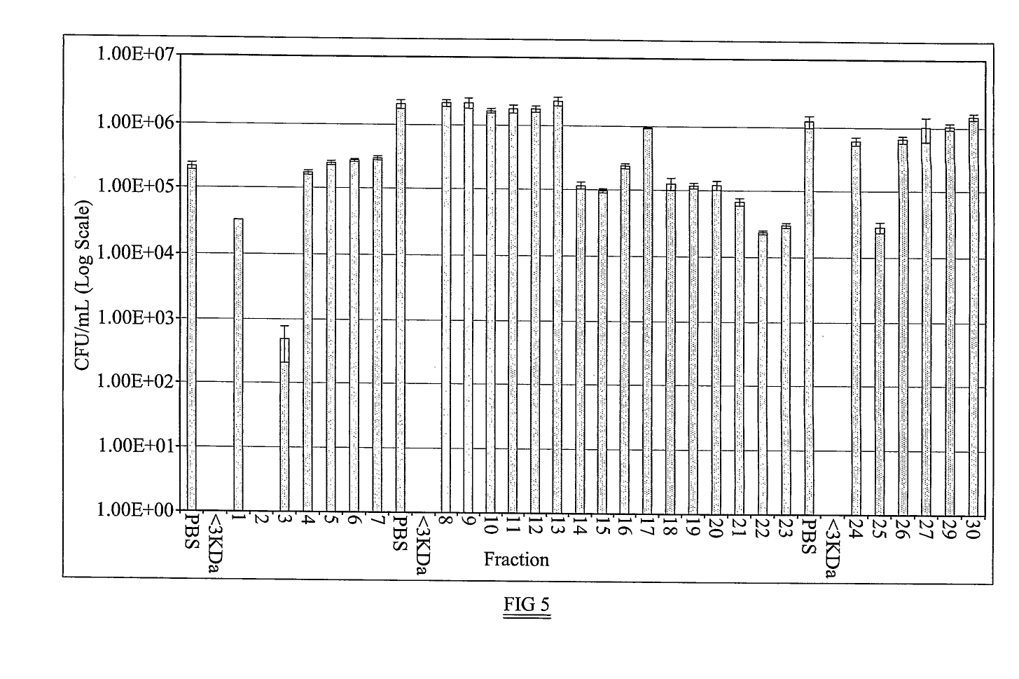

FIG. 5 is a graph showing the activity profile of the less than or equal to 3 kDa fraction after anion exchange HPLC;

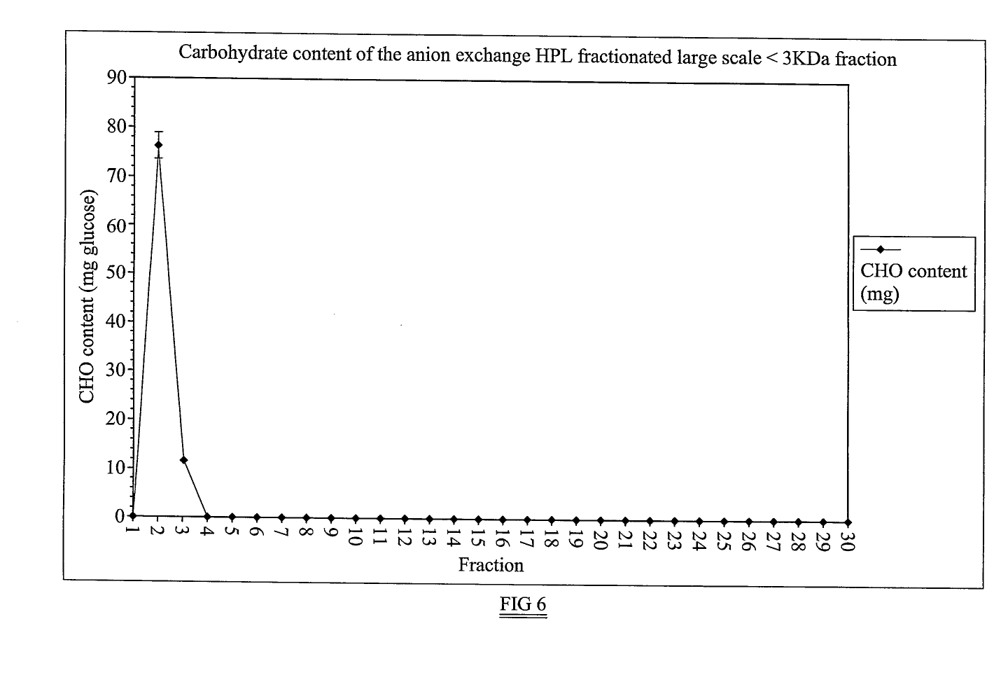

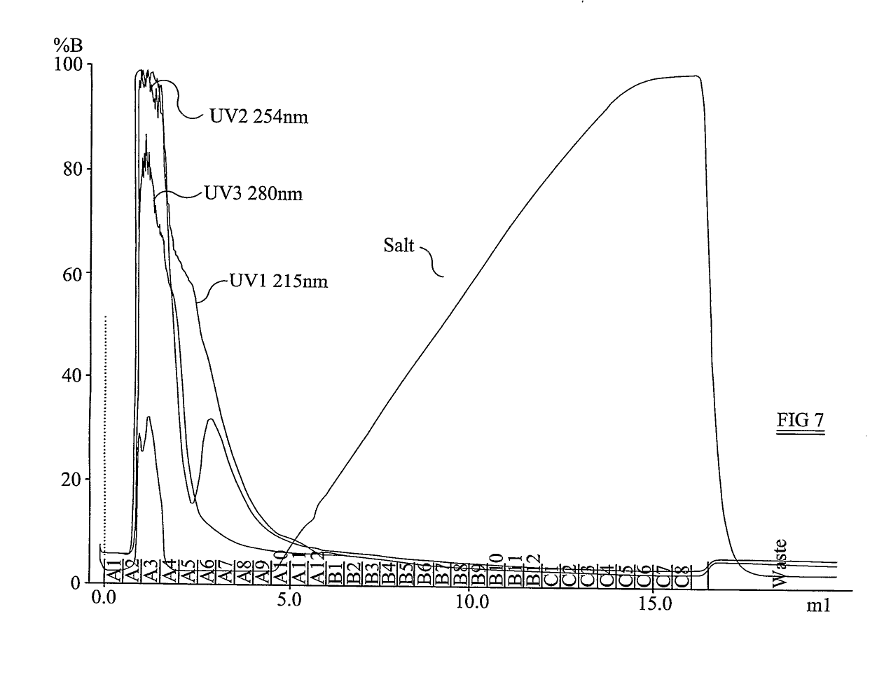

FIG. 6 is a graph showing the carbohydrate content of the anion exchange HPLC fractionated large scale less than or equal to 3 kDa fraction;

FIG. 7 is a cation exchange HPLC chromatogram of bulk isolated less than or equal to kDa fraction anion exchange flow through; and

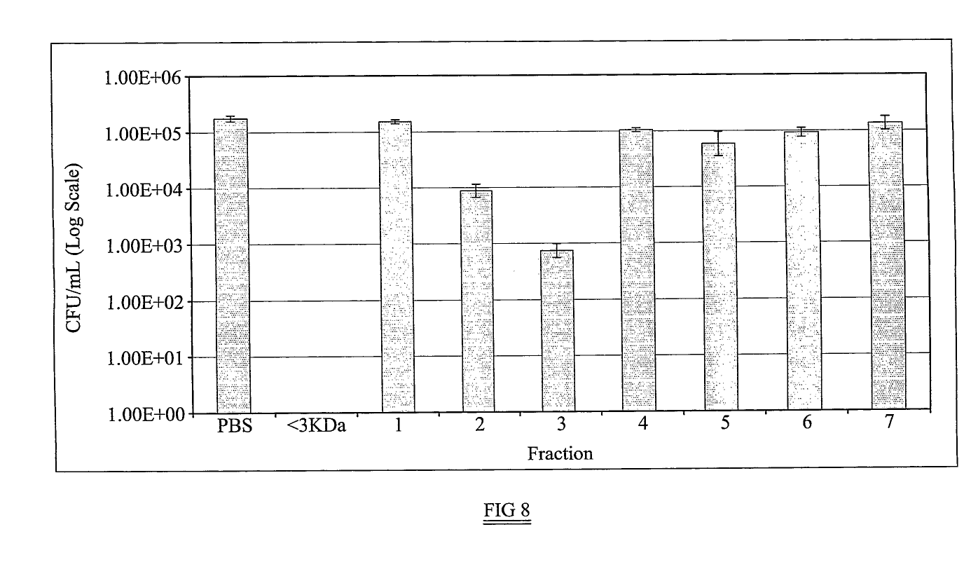

FIG. 8 is a graph showing the activity profile of the less than or equal to 3 kDa fraction flow though after cation exchange HPLC.

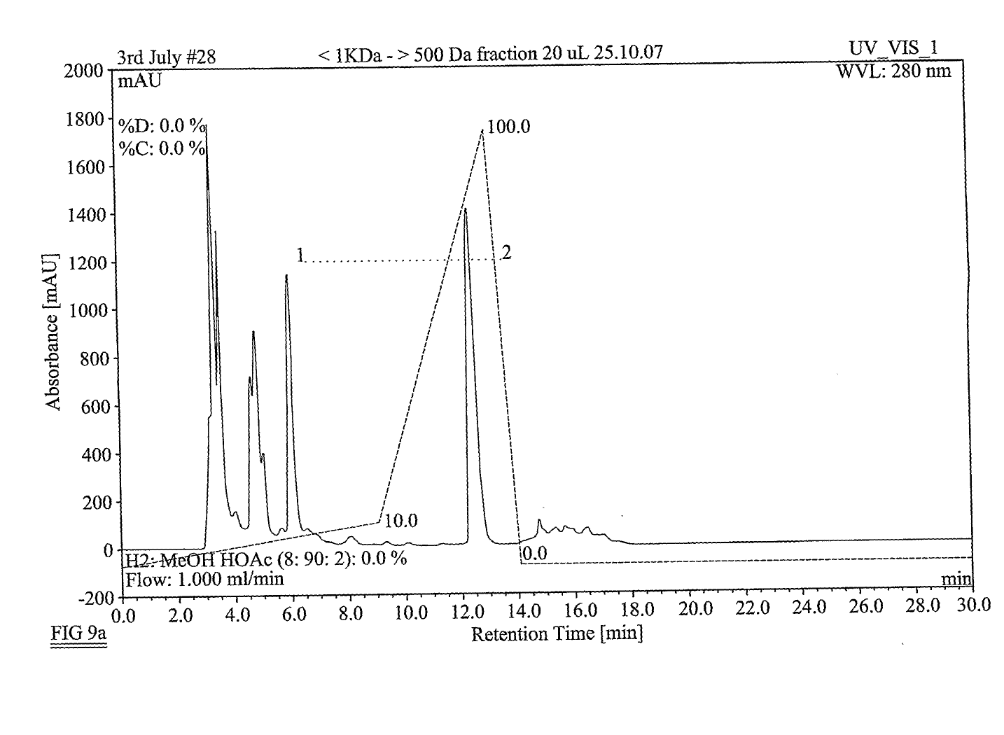

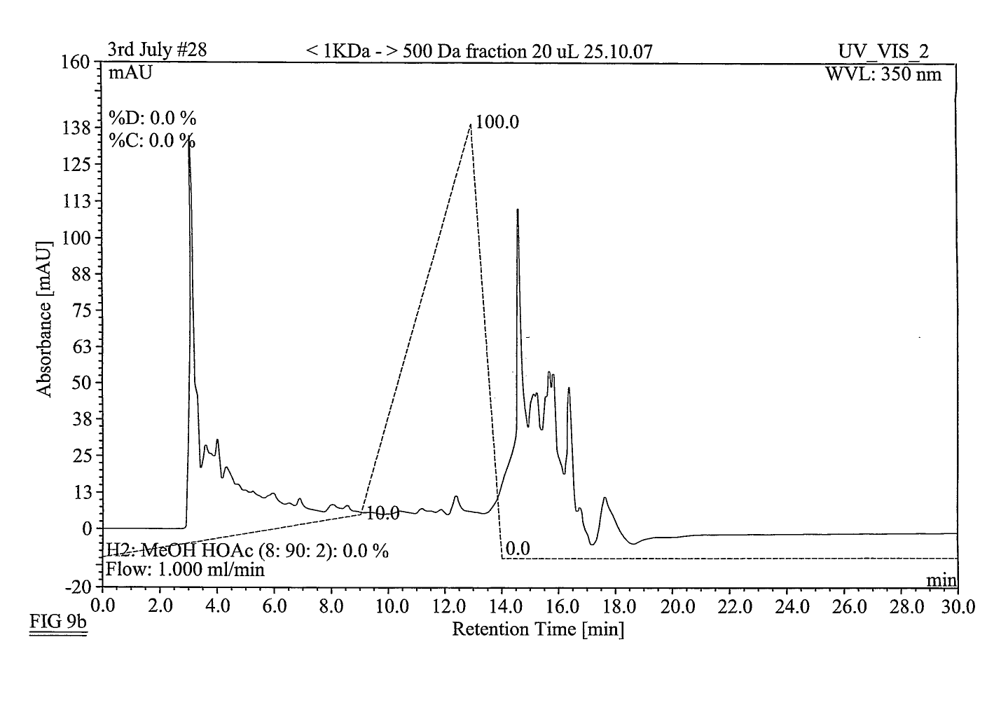

FIG. 9a is a HPLC chromatogram of the less than or equal to 1 kDa to greater than or equal to 500 Da fraction on a Gemini C₁₈ reversed phase column. UV detection at 280 nm 1, 2: peaks 1 and 2;

FIG. 9b is a HPLC chromatogram of the less than or equal to 1 kDa to greater than or equal to 500 Da fraction on a Gemini C₁₈ reversed phase column. UV detection at 350 nm;

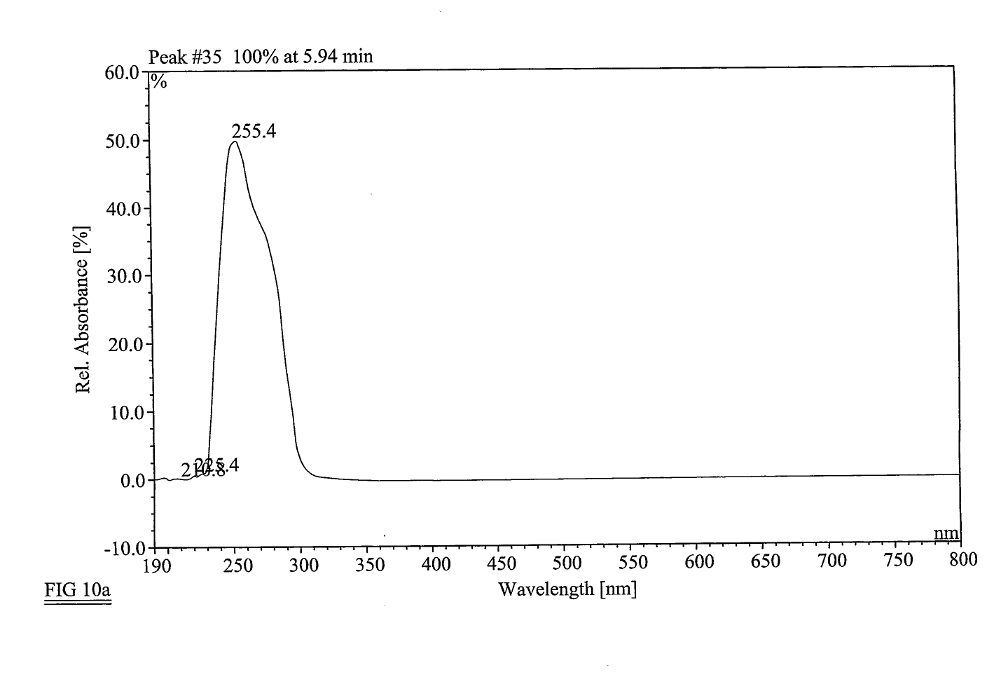

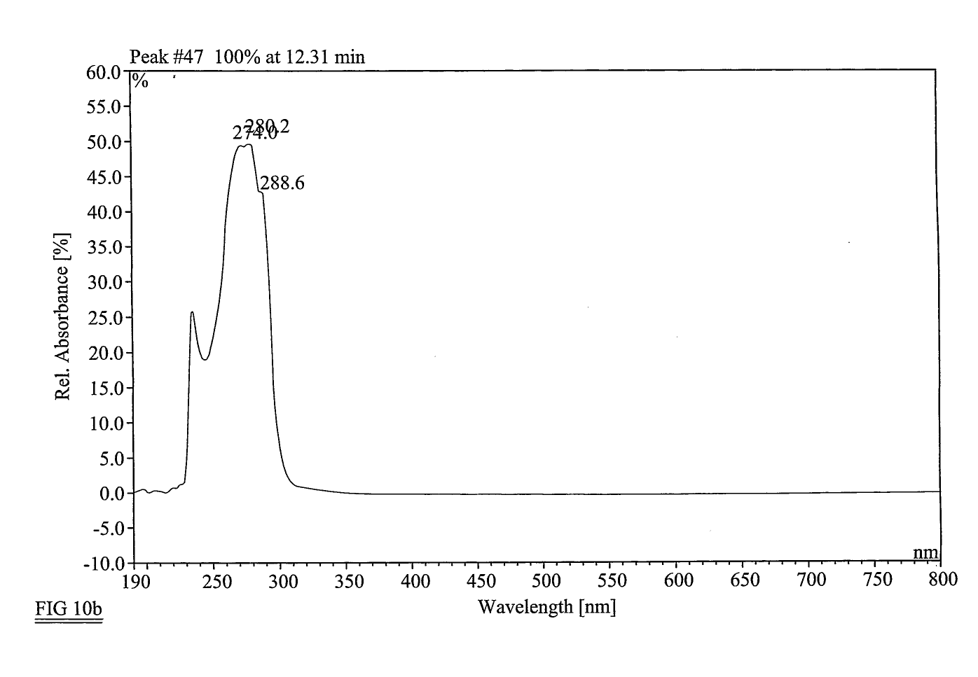

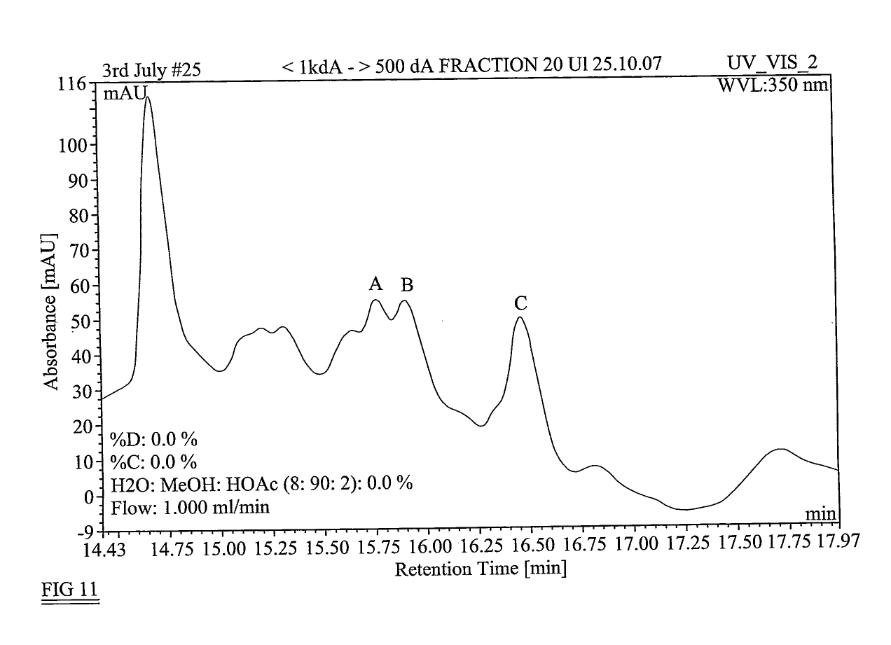

FIG. 10a is a UV spectrum of peak 1 at 280 nm;

FIG. 10b is a UV spectrum of peak 2 at 280 nm;

FIG. 11 is a close up view of the components eluting between 14 to 17 minutes with detection at 350 nm. A-C: peaks A-C;

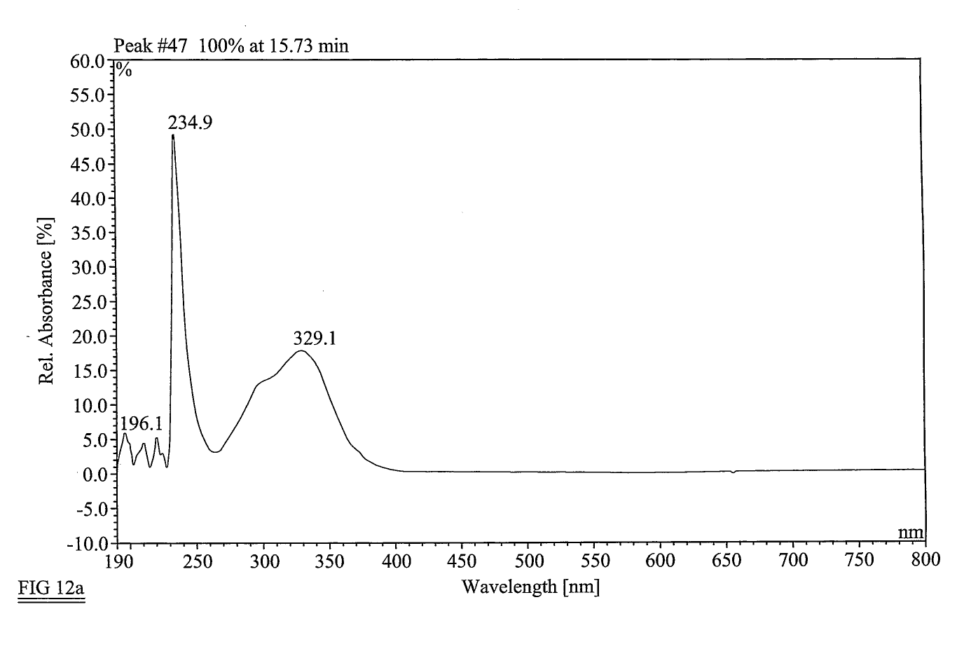

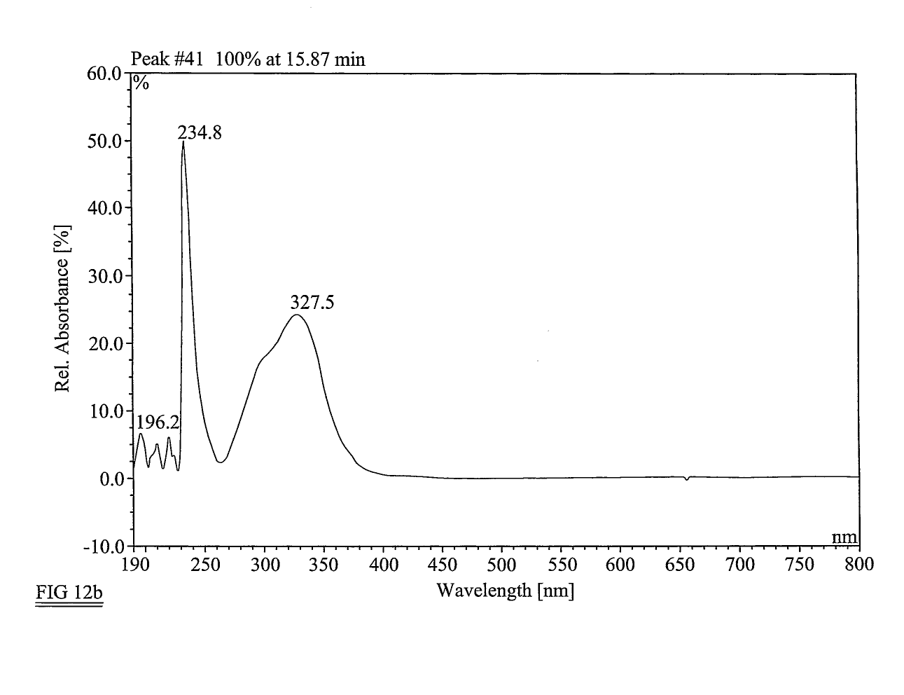

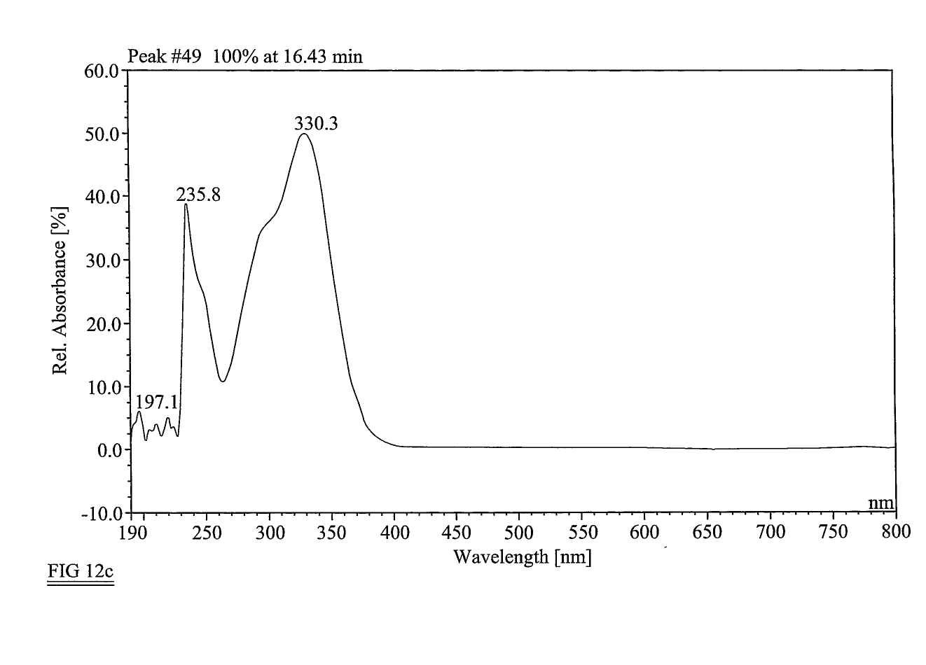

FIG. 12a is a UV spectrum of peak A at 350 nm;

FIG. 12b is a UV spectrum of peak B at 350 nm;

FIG. 12c is a UV spectrum of peak C at 250 nm;

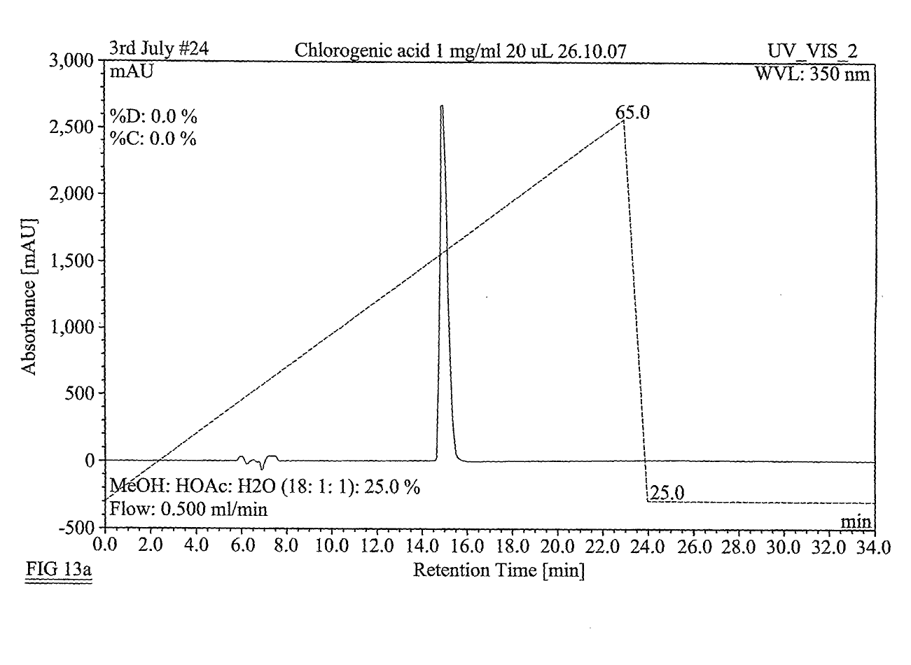

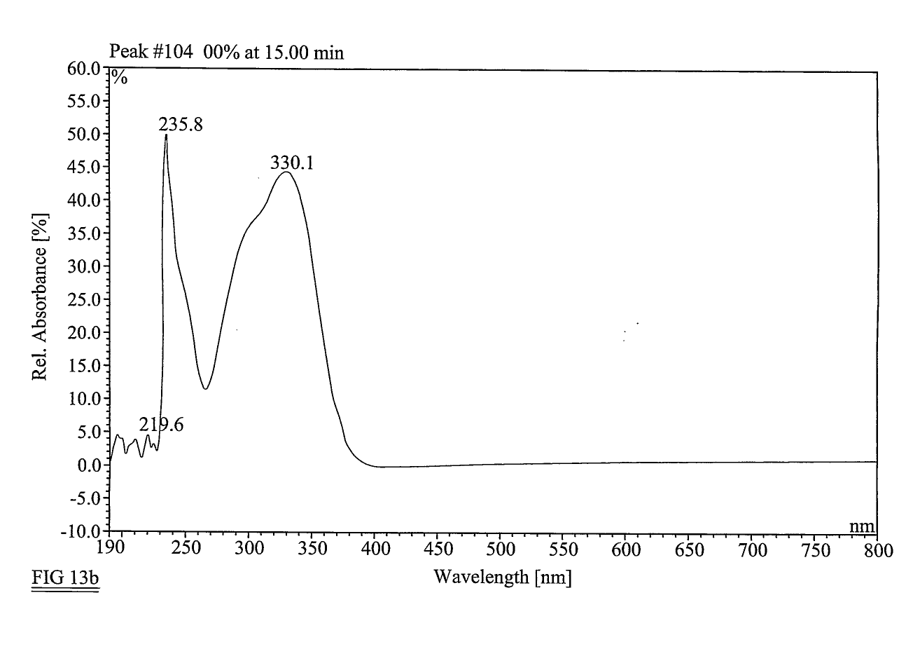

FIG. 13a is a HPLC chromatogram of chlorogenic acid marker at 350 nm; and

FIG. 13b is a UV spectrum of chlorogenic acid marker at 350 nm.

The inventors conducted studies to investigate what effect potato juice has on Helicobacter pylori. In particular, an investigation was conducted to determine whether potato juice was able to disrupt adhesion of H. pylori to gastric epithelial cells. The rationale being that disruption of H. pylori colonisation would lead to a clearance of the organism and a reduction in host tissue damage and symptoms. Further investigations were conducted to establish that the anti-adhesive properties of the potato juice are located in a less than or equal to 3 kDa fraction, more specifically an equal to or greater than 500 Da to less than or equal to 1 kDa soluble fraction derivable from potato juice that appears to be neutrally charged.

### Preparation of Extracts.

Potato juice was generated by first homogenizing peeled King Edwards potatoes in a food blender, sieve filtering the ground potatoes and centrifuging the soluble fraction at 8000 to 3000 rpm for 5 to 20 minutes. The solution was then filtered using a 0.22 μm filter and the extract was stored at −80° C. until required.

Freshly prepared crude potato juice was fractionated by centrifugal filtration in VIVA Spin 20 centrifugal filters (MWCO 3 kDa) at 3,660 rpm, 3 times for 60 minutes. The collected less than or equal to 3 kDa fraction was stored at −80° C. until required for further analysis.

Additionally, a large scale less than or equal to 3 kDa potato juice fraction was prepared for isolation of the anti-adhesive components. Washed and peeled potatoes (8.66 Kg) were ground and filtered to give 2,215 mL of crude potato juice which after fractionation by centrifugal filtration gave 853 mL of the less than or equal to 3 kDa fraction. This was lyophilised and resuspended in water (25 mL) to give a yellow-red viscous liquid. The concentrated fraction was stored at −80° C. until required for further analysis.

### EXAMPLE 1

The effect of potato juice on the adhesion of H. pylori was determined using a modification of the method described by Bennett and Roberts, 2005 (19) in which potato juice, or fractions thereof, were included either before or after incubation of H. pylori with AGS cells.

AGS cells were fixed to a 96 well plate using paraformaldehyde and glycine. H. pylori cultures were adjusted to an OD (600)=0.25 and mixed with the sample extract in a 1:1 ratio. 100 μL aliquots were added to each well and spun for 1,000 rpm for 10 minutes followed by microaerophilic incubation for 90 minutes at 37° C. The liquid was removed from the well and the bound cells were treated with 1% saponin. Samples were then serially diluted and processed for viable counts via standard procedures.

### Results.

The experiment showed that the less than or equal to 3 kDa fraction had a dramatic effect on the binding of H. pylori to AGS cells, as shown in FIG. 1 of the accompanying drawings.

Following binding of H. pylori cells to AGS cells, serial dilutions of the less than or equal to 3 kDa fraction were added. Subsequently, the number of bound bacteria was determined, with the number of bacteria bound in the PBS control expressed as 100%. Concentration is undiluted less than 3 kDa fraction. Titration showed a dose dependent effect (see FIG. 2). The results illustrated in FIGS. 1 and 2 were obtained following addition of the fraction after bacterial attachment but excitingly, the affect of the less than or equal to 3 kDa fraction on the binding of H. pylori to AGS cells was found to work whether the fraction was added at the same time as the H. pylori to the host cells or if added after bacterial attachment (as evidenced by a displacement assay). This indicates that the anti-adhesive component present in the less than or equal to 3 kDa fraction is capable of not only inhibiting binding but also capable of removing bound H. pylori from AGS cells, thus offering the possibility of removing bound H. pylori infected hosts thereby promoting eradication.

### EXAMPLE 2

The effect of heat and proteinase K digest on the activity of potato juice was investigated. The results showed that the anti-adhesive properties were unaffected by heat treatment and incubation with proteinase K (PK) did not appear to exhibit any notable effect on the activity of the extract (FIG. 3). Proteinase K is a non specific protease that cleaves peptide bonds at the carboxylic sides of aliphatic, aromatic or hydrophobic amino acids. In terms of the latter treatment, this suggests that the active components are not proteinaceous in nature.

### EXAMPLE 3

Anion and cation HPLC fractionations of the less than or equal to 3 kDa fraction were carried out to purify the anti-adhesive component of the fraction.

Proteins and other biomolecules possess a property known as a PI value which is the pH at which the molecule carries no net charge. At a pH above the PI value the molecule is negatively charged whereas at a pH below the PI value the molecule is positively charged. This property can be exploited during ion exchange chromatography. Basically ion exchange involves eluting an extract on a positively charged column (anion exchange) or on a negatively charged column (cation exchange), initially with a buffer such as Tris or MES to elute unbound material and then introducing a linear salt gradient to displace molecules bound to the column at the respective pH. In order to bind the molecules must possess a net charge opposite to that of the column at the pH used.

All separations were done on an ETTAN LC (Amersham Biosciences) equipped with a UV detector (215, 254 and 280 nm) and a conductivity recorder. The results were analysed using Unicorn™ software.

For the anion exchange fractionation, the column used was MonoQ HR 5/5 (1 mL volume), charged group: O—CH₂—CHOH—CH₂—O—CH₂—CHOH—CH₂—N⁺(CH₃)₃. Mobile phase: Buffer A: 20 mM Tris-HCl (pH 8), Buffer B: 20 mM Tris in 2 M NaCl (pH 8). The column was eluted with 3.5 column volumes of 100% Buffer A to remove unbound material before introduction of a linear gradient mode going from 0% B to 100% B over 10 column volumes to displace bound material. The flow rate was between 0.25 to 0.4 mL.min⁻¹. Between 10 and 20, 250 μL aliquots of each fraction (in buffer A) were delivered via a 500 μL injection loop. Then, 30, 0.5 mL fractions were collected for each run and the fraction replicates were pooled, lyophilised and resuspended in 500 μL before assaying for anti-adhesive activity. Prior to assaying fractions 8 to 30 were desalted via dialysis against distilled water in Spectra/Por® dialysis tubes (MWCO 1 kDa). Desalted fractions were lyophilised and resuspended in 500 μL water.

After separation by anion exchange the results showed that the UV absorbing material that eluted in fractions 2 to 5 (FIG. 4) represents the “flow through” and at pH 8 this material is either neutral or positively charged. The material eluting later on between fractions 10 to 17 has bound to the column so at pH 8 these components carry a net negative charge. Activity screening showed that the active components were present in the flow through and therefore are either neutral or positively charged at pH 8 (FIG. 5). Sugar analysis (using the phenol-sulphuric acid method originally described by Dubois et al (20) modified to facilitate analysis in a 96 well plate reader) showed that after anion exchange all the carbohydrate eluted in the flow through (FIG. 6). These results show that anion exchange has separated the active components from the negatively charged material at pH 8.

The anion exchange flow through (fractions 2 to 7) was isolated in bulk and the resulting fraction was separated by cation exchange using the same conditions as employed for the anion exchange experiments. The HPLC chromatogram showed that all of the UV absorbing material did not stick to the column as it eluted before introduction of the salt gradient (FIG. 7). Likewise after cation exchange the anti-adhesive components appear to be still present in the flow through indicating their neutral nature at pH 8 (FIG. 8). Thus after anion exchange on a MonoQ column followed by cation exchange of the active fractions on a MonoS column the anti-adhesive components appear to carry no net charge and thus are neutral at pH 8. It is clear that at pH 8 some purification has occurred as the activity has been separated from a significant amount of negatively charged material via anion exchange.

### EXAMPLE 4

Phytochemical analysis was carried out in relation to the less than or equal to 3 kDa fraction:

TLC analysis of the fraction showed the presence of phenolic glycosides, nitrogenous compounds and a number of unknowns as shown in Table 1 below. Furthermore, TLC separation of the extract alongside markers representing the 20 known protein amino acids suggested the presence of threonine, valine and leucine or isoleucine or methionine.

TABLE 1
TLC data on less than or equal to 3 kDa fraction

| TLC system | Chromogenic reagent | Compound class | Result |
|---|---|---|---|
| 1-D TLC on silica gel in | Iodoplantinate, Marquis, | Alkaloids | Negative |
| MeOH:NH₄OH (200:3), | Ehrlich's | | |
| CHCl₃:MeOH:2% | | | |
| NH₄OH (70:30:5) | | | |
| 2-D TLC on cellulose in | Folin-Ciocalteu | Phenol glycosides | Positive |
| (1) BAW (4:1:5, upper | | | |
| layer) and (2) 15% HOAc | | | |
| 2-D TLC (acid hydrolysed | Folin-Ciocalteu | Phenol aglycones | Positive |
| less than or equal to 3 KDa | | | |
| fraction) on silica gel in | | | |
| (1) 10% HOAc in CHCl₃ (2) | | | |
| EtOAc:benzene (45:55) | | | |
| 1-D TLC on cellulose in | Folin-Ciocalteu | Flavonoid aglycones | Negative |
| forestall | | | |
| 1-D TLC on silica gel in | Ninhydrin + heating @ | Nitrogenous compounds | Positive |
| CHCl₃:MeOH:NH₄OH:H₂O | 100° C. | other than alkaloids | |
| (70:26:2:2) | | (amines, amino acids) | |

The crude or partially purified less than or equal to 3 kDa fraction may be further processed according to the invention to provide a functional food product that can be used to prevent or treat gastric and duodenal ulcers and carcinomas.

In this respect, whilst the consumption of raw potato juice may alleviate the symptoms of gastritis it would clearly be impossible to maximize health benefits in this way due to the frequency and quantity of potato juice that would need to be consumed. The identification and isolation of a less than or equal to 3 kDa fraction according to the present invention enables a concentrated amount of the bioactive part of the juice to be introduced into a suitable product (“functional food”) which may then be consumed as required to obtain the desired health benefit.

### EXAMPLE 5

### Preparation of a Powder of a 3 kDa Fraction of Potato Juice

A less than or equal to 3 kDa extract of homogenized potato juice was prepared as described above and freeze-dried to form a powdered extract according to the invention.

### EXAMPLE 6

### Preparation of a Powder Mix of a 3 kDa Fraction of Potato Juice

3.0 g of freeze dried powdered extract (Example 4) was mixed with 0.5 g powdered citric acid, 26.3 g of granulated sugar and 0.2 g of a standard spray-dried mix of flavouring.

This mixture represents a free-flowing powder formulation (containing 3.0 g of the active fraction) that is suitable for packaging in a sachet. The powder mix may be diluted to taste and drunk when required by a subject suffering from gastritis.

### EXAMPLE 7

### Preparation of an Orange Drink for Use According to the Invention

(a) 100 ml of crude preparation was mixed with 100 mls of double concentrate orange juice (orange juice concentrate diluted in water to double strength);
(b) 3.5 g of freeze-dried powder was dissolved in 100 mls of orange juice (or alternatively with orange juice concentrate and water).

The orange drink preparations (a) or (b) may be consumed by a subject immediately, refrigerated for later consumption or sealed in a bottle or carton for a longer shelf life.

It will be appreciated that orange juice may be readily substituted with a palatable alternative, such as milk.

### EXAMPLE 8

### HPLC Finger Print Analysis of a Less than or Equal to 1 kDa to Greater than or Equal to 500 Da Fraction

An anti-adhesive fraction, that is, a fraction with less than or equal to 1 kDa to greater than or equal to 500 Da fraction, was analysed by reversed phase HPLC to provide a separation that would be directly suitable for LC-MS analysis without the presence of salt as is the case with the HPAEC HPLC chromatogram. The HPLC column used was a Gemini C₁₈ 250×4.6 mm i.d column with a particle size of 5 μm utilising a programme designed to separate the free phenolic compounds in potatoes (21). The mobile phase consisted of A=water:methanol:acetic acid (88:10:2) and B=water:methanol:acetic acid (8:90:2) and involved an increasing gradient of B to 10% over 9 minutes followed by a sharp increase to 100% B between 9 to 13 minutes before returning to the initial conditions. The results below show a typical trace obtained for the less than or equal to 1 kDa to greater than or equal to 500 Da fraction (FIG. 9a). When monitored at 280 nm there were several absorbing peaks but notably two, 1 and 2 appeared to have separated (FIG. 9a). The UV spectrum of peak 1 and 2 are shown in FIGS. 10a and 10b respectively. Both these components are aromatic in nature and could be purines, pyrimidines or aromatic amino acid residues. At 350 nm the HPLC chromatogram was different (FIG. 9b) with components eluting between 14 to 17 minutes. A close up of this region (FIG. 11) revealed several peaks. Although not completely resolved peaks A-C had UV spectra diagnostic of caffeic acid derivatives (FIGS. 12a to 12c), phenolic phenylpropanoids belonging to the same family as chlorogenic acid (FIGS. 13a and b), the predominant phenol in potatoes. No prominent peak was observed at 15 minutes which is when the chlorogenic acid marker eluted (FIG. 13 a,b).

Therefore in summation there are at least 3 caffeic acid derivatives present in minor concentrations in the less than or equal to 1 kDa to greater than or equal to 500 Da fraction, none of which are chlorogenic acid. Furthermore there are several aromatic components in the fraction of which two appear to have separated.

### REFERENCES

1. Blaser, M. J. (1998) Helicobacter pylori and gastric diseases. Biomed J. 316: 1507-1510.
2. Uemura, N., Okamoto, S., Yamamoto, S., Matsumura, N., Yamaguchi, S., Yamakido, M., Taniyama, K., and Sasaki, N., Schlemper, R. J. (2001) Helicobacter pylori infection and the development of gastric cancer. N. Engl. J. Med. 345: 784-789.
3. Guruge, J. L., Falk, P. G., Lorenz, R. G., Dans, M., Wirth, H. P., Blaser, M. J., Berg, D. E., and Gordon, J. I. (1998) Epithelial attachment alters the outcome of Helicobacter pylori infection. Proc, Natl, Acad, Sci. USA 95: 3925-3930
4. Censini, S., Stein, M., and Covacci, A. (2001) Cellular responses induced after contact with Helicobacter pylori. Curr. Opin. Microbiol. 4: 41-46
5. Calvert, X., Ducons, J., Bujanda, L., Bory, F., Montserrat, A., and Gisbert, J. P. (2005) Seven versus ten days of rabeprrazole triple therapy for Helicobacter pylori eradication: a multicenter randomised trial. Am. J. Gastroenterol 100: 1696-1701
6. Goh, K. L. (2002) Update on the management of Helicobacter pylori infection, including drug resistant organisms. J Gastroenterol Hepatol 17: 482-487.
7. Kamiji, M. M., and de Oliveira R. B. (2005) Non-antibiotic therapies for Helicobacter pylori infection. Eur J Gastroenterol Hepatol 17: 973-981.
8. Galan, M. V., Kishan, A. A., Silverman, A. L., 2004. Oral broccoli sprouts for the treatment of Helicobacter pylori infection: a preliminary report. Digestive Diseases and Sciences 49, 1088-1090.
9. Fahey, J. W., Haristoy, X., Dolan, P. M., Kensler, T. W., Scholtus, I., Stephenson, K. K., Talalay, P., Lozneiwski, A., 2002. Sulforaphane inhibits extracellular, intracellular, and antibiotic-resistant strains of Helicobacter pylori and prevents benzo[a]pyrene induced stomach tumors. PNAS 99, 7610-7615.
10. Adeniyi, B. A., Anyiam, F. M., 2004. In vitro anti-Helicobacter pylori potential of methanol extract of Alluim ascalonicum Linn. (Liliacaea) leaf: susceptibility and effect on urease activity. Phytotherapy Research 18, 358-361.
11. Lin, Y. T., Kwon, Y. I., Labbe, R. G., Shetty, K., 2005 Inhibition of Helicobacter pylori and associated urease by oregano and cranberry phytochemical synergies. 71, 8558-8564.
12. Mahady, G. B., Pendland, S. L., Stoia, A., Chadwick, L. R., 2003. In vitro susceptibility of Helicobacter pylori to isoquinoline alkaloids from Sanguinaria Canadensis and Hydrastis canadensis. Phytotherapy Research 17, 217-221.
13. Wang, Y-C., Huang, T-L., 2005. Anti-Helicobacter pylori activity of Plumbago zeylanica L. FEMS Immunology and Medical Microbiology 43, 407-412.
14. Stamatis G., Kyriazopoulos, P., Golegou, S., Basayiannis, A., Skaltsas, S., Skaltsa, H., 2003. In vitro anti-Helicobacter pylori activity of Greek herbal medicines. Journal of Ethnopharmacology 88, 175-179.
15. Matsubara, S., Shibata, H., Ishikawa, F., Yokokura, T., Takahashi, M., Sugimura, T., Wakabayashi, K., 2003. Suppression of Helicobacter pylori-induced gastritis by green tea extract in Mongolian gerbils. Biochemical and Biophysical Research Communications 310, 715-719.
16. O′Mahony, R., Al-Khtheeri, H., Weerasekera, D., Fernando, N., Vairi, D., Holton, J., Basset, C., 2005. Bactericidal and anti-adhesive properties of culinary and medicinal plants against Helicobacter pylori. World Journal of Gastroenterology 11, 7499-7507.
17. Deans, S. G., Ritchie, G., 1987. Antibacterial properties of plant essential oils. International Journal of Food Microbiology 5, 165-180.
18. Chou, L-S., Weimer, B., 1999. Isolation and characterization of acid- and bile-tolerant isolates from strains of Lactobacillus acidophilus. Journal of Dairy Science 82, 23-31.
19. Bennett, H., and Roberts, I. S. (2005) Identification of a new sialic acid-binding protein in Helicobacter pylori. FEMS. Immunol, Med. Microbiol. 44: 163-169.
20. Dubois, M., Gilles, K. A., Hamilton, J. K., Rebers, P. A., Smith, F., 1956. Colorimetric method for determination of sugars and related substances. Analytical Chemistry 28, 350-356.
21. Verde-Mendez, C, Delgado, M. A. R., Rodriguez, E. M. R., Romero, C. D., 2004. Content and free phenolic compounds in cultivars of potatoes harvested in Tenerife (Canary Islands). J. Agric. Food Chem. 52, 1323-1327.

1. A composition for use in the prevention or treatment of gastrointestinal disease comprising a therapeutically effective amount of a less than or equal to 4 kDa soluble fraction derivable from potato juice.

2. A composition according to claim 1, wherein the soluble fraction derivable from potato juice is a less than or equal to 3 kDa fraction.

3. A composition according to claim 2 wherein the soluble fraction derivable from potato juice is between 1 and 3 kDa fraction.

4. A composition according to claim 1 wherein the soluble fraction derivable from potato juice comprises a therapeutically effective amount of equal to or greater than 500 Da to less than or equal to 1 kDa.

5. A composition according to claim 4 wherein the greater than 500 Da to less than or equal to 1 kDa soluble fraction comprises three or more caffeic acid derivatives and two aromatic derivatives.

6. A composition according to claim 1 wherein the fraction is neutral at pH 8.

7. A composition according to claim 1 wherein the potato juice fraction is subjected to anion HPLC at pH 8 to bind negatively charged components, the flow through collected and then subjected to cation HPLC at pH 8 to bind positively charged components, eluting the positively bound components and collecting the eluted fraction.

8. A composition according to claim 1 wherein the potato juice fraction includes at least one oligosaccharide.

9. A composition according to claim 1 wherein the potato juice fraction includes phenol glycosides, phenol aglycones and/or nitrogenous compounds other than alkaloids.

10. A composition according to claim 1 in powder form.

11. A composition according to claim 10 wherein the powder comprises a spray-dried or freeze-dried fraction.

12. A composition according to claim 1 further comprising at least one of flavourings, sugars, sweetener, anti-oxidants, minerals or vitamins.

13. A composition according to claim 1 suitable for the prevention and/or treatment of gastric or duodenal ulcers and gastric or duodenal carcinomas.

14-17. (canceled)

18. A method for the treatment of gastrointestinal disease comprising administering to a subject in need of such treatment a therapeutically effective amount of a less than or equal to 4 kDa soluble fraction derivable from potato juice.

19. A method according to claim 18 comprising administering to a subject in need of such treatment a therapeutically effective amount of an equal to or greater than 500 Da to less than or equal to 1 kDa soluble fraction derivable from potato juice.

20. A nutritional product for use in the prevention or treatment of gastrointestinal disease comprising a therapeutically effective amount of a less than or equal to 4 kDa soluble fraction derivable from potato juice.

21. A nutritional product according to claim 20 comprising a therapeutically effective amount of an equal to or greater than 500 Da to less than or equal to 1 kDa soluble fraction derivable from potato juice.

22. A nutritional product according to claim 20 in the form of a beverage or drink.

23. A nutritional product according to claim 20 in the form of a dairy product.

24. A nutritional product according to claim 20 comprising between 1 g/100 ml and 30 g/100 ml of the fraction.

25. A nutritional product according to claim 20 in the form of a powder or powder mix.

26. A nutritional product according to claim 20 in the form of a food bar or other solid food stuff.

27. A soluble fraction derivable from potato juice comprising an amount of equal to or greater than 500 Da to less than or equal to 1 kDa wherein the soluble fraction comprises three or more caffeic acid derivatives and two aromatic derivatives.
